(19) United States
(12) Patent Application Publication
(10) Pub. No.: US 2023/0329874 A1
(43) Pub. Date: Oct. 19, 2023

(54) TIBIAL COMPONENT OF A TOTAL KNEE PROSTHESIS

(71) Applicants: Diego ZAMAGNI, Savignano Sul Rubicone (IT); Mohamed Mahmoud Abouelfettouh Abdelgelil ELFEKKY, Sharjah (AE); Samih ALTARABICHI, Dubai (AE)

(72) Inventors: Diego ZAMAGNI, Savignano Sul Rubicone (IT); Mohamed Mahmoud Abouelfettouh Abdelgelil ELFEKKY, Sharjah (AE); Samih ALTARABICHI, Dubai (AE)

(21) Appl. No.: 18/300,136

(22) Filed: Apr. 13, 2023

(30) Foreign Application Priority Data
Apr. 15, 2022 (IT) 102022000007586

(51) Int. Cl. A61F 2/38

(52) U.S. Cl. CPC A61F 2/389; A61F 2002/30245

(57) ABSTRACT

A tibial component of a total knee prosthesis which includes a base element provided with a medial portion and a lateral portion, which can be fixed on the proximal surface of the tibia-; The base element has shape and dimensions that are complementary to those of at least one protective insert (one single insert or two separate inserts: one medial and one lateral).
—— In particular, the medial portion is provided with coupling elements for coupling by pressure to the insert (a configuration generally termed “fixed insert”).
——— By contrast, the at least one insert is resting on the lateral portion.

### CROSS-REFERENCE TO RELATED APPLICATIONS

This application claims the benefit of Italian patent application 102022000007586, filed on 15 Apr. 2022, the disclosure of which is incorporated herein by reference in its entirety.

### TECHNICAL FIELD

The present disclosure relates to a tibial component of a total knee prosthesis.

### BACKGROUND

It is known that in the knee joint, the cartilage that covers the end part of the bone of the femur, the tibial plateau and the patella, acts as a cushion, facilitating a fluid and uniform movement of the bones against each other.

However, when this cartilage wears away and the bones start to rub against each other, this condition can cause an acute pain which reduces the functionality of the knee.

The aim of total knee prosthesis surgery is to substitute the damaged bone and cartilage with a prosthesis in order to recreate the surface of the knee joint. These new surfaces are designed to replicate the functionality of a healthy knee and at the same time prevent the pain caused by the mutual friction of the damaged bones.

Currently, substantially three main types of total knee prosthesis exist: “cruciate-retaining (CR) total knee arthroplasty (TKA)” prostheses, which conserve the posterior cruciate ligament, “posterior-stabilized (PS) total knee arthroplasty (TKA)” prostheses, which entail the removal of both cruciates, “bicruciate retaining (BCR) total knee arthroplasty (TKA)” prostheses, which make it possible to preserve both cruciates.

Independently of the type of total knee prosthesis used, they comprise substantially a first element, made of metal, to be anchored to the distal end of the femur (hence the name femoral component), a second element, also made of metal, to be anchored to the proximal end of the tibia (called tibial component), and a single insert or two separate inserts (one medial and one lateral) interposed between them, usually made of polymeric material, which substantially constitute/s the track for the femoral component to slide on the tibial component (and a form of shock-absorbing meniscus cushion for the prosthetic joint).

Such insert can be of the fixed or movable type.

In the first case, the insert is fixed to the tibial component with means for locking by pressure.

This approach aims to limit any macro/micro movement of the insert with respect to the tibial component, thus ensuring a stable joint and eliminating the risk of dislocation of said insert.

However, in order to ensure that the joint has the necessary and physiological mobility, the insert has a substantially flat lateral joint surface that, by interfacing with a convex femoral surface, leads to a greater risk of wear of the prosthetic insert.

When a movable insert is used instead, the insert has total or partial freedom of movement with respect to the tibial component. Furthermore, the insert has a substantially concave surface, so as to maximize the area of contact between said insert and the (convex) femoral component and reduce the risk of wear of the polyethylene, in this manner overcoming the mobility/congruence conflict typical of fixed inserts.

However, the absence of a locking mechanism brings a non-negligible risk of dislocation of the insert.

It needs to be emphasized that in a total reconstruction, or Total Knee Arthroplasty (TKA), these two philosophies turn out to be exclusive and solitary, in that they offer the same approach (fixed or movable insert) to both the lateral and medial compartments, taking the knee as a single, uniform and single joint.

In reality, the human knee has a marked compartmental disparity, in both anatomical and kinematic terms.

In fact, the medial compartment has a concave tibial condyle which, together with a rigid meniscus, ensures a stable medial joint during the ROM (Range Of Motion) (average A/P anteroposterior translation = +/- 1.5 mm), while the lateral compartment is characterized by a convex tibial condyle which, together with a movable meniscus, ensures ample lateral mobility (average A/P anteroposterior translation = 18 mm). Furthermore, in deep flexion, the lateral femoral condyle rolls so far rearward that it requires a significant translation of the lateral meniscus.

Trying to faithfully reproduce this compartmental disparity with a single solution (with a fixed or movable insert) is impossible and conceptually wrong; fixed-insert prostheses inexorably fail to recreate lateral mobility, and movable-insert prostheses inexorably fail to recreate the medial stability of a healthy knee joint.

It is therefore evident that each of the two approaches brings with it major advantages, but also entails significant drawbacks, which means that a total knee prosthesis presents kinematic and proprioceptive performance levels that are decidedly inferior to and different from the performance of a healthy knee joint or an unicompartmental partial arthroplasty (in which only one of the two portions of the knee, the medial portion or the lateral portion, is reconstructed).

### SUMMARY

The aim of the present disclosure is to solve the above mentioned drawbacks, by providing a tibial component of a total knee prosthesis that makes it possible to recreate the physiological medial stability and the physiological lateral mobility observed in the normal kinematic arrangement of healthy knee joints.

Within this aim, the disclosure provides a tibial component of a total knee prosthesis that makes it possible to reduce the risk of dislocation of the at least one insert to the minimum.

the present disclosure also provides a tibial component of a total knee prosthesis that makes it possible to reduce the risk of prosthetic instability to the minimum over time.

the disclosure further provides a tibial component of a total knee prosthesis that makes it possible to reduce phenomena of wear of the total knee prosthesis to the minimum.

The present disclosure provides a tibial component of a total knee prosthesis that is of low cost, easily and practically implemented and safe in use.

This aim and these and other advantages which will become better apparent hereinafter are achieved by providing a tibial component of a total knee prosthesis, comprising a base element provided with a medial portion and a lateral portion, which can be fixed on the proximal surface of the tibia, characterized in that said base element has shape and dimensions that are complementary to those of said at least one protective insert, said medial portion being provided with coupling means by pressure to said at least one insert, the at least one insert resting on said lateral portion.

### BRIEF DESCRIPTION OF THEDRAWINGS

Further characteristics and advantages of the disclosure will become better apparent from the detailed description that follows of a preferred, but not exclusive, embodiment of the tibial component of a total knee prosthesis, according to the disclosure, illustrated by way of non-limiting example in the accompanying drawings wherein:

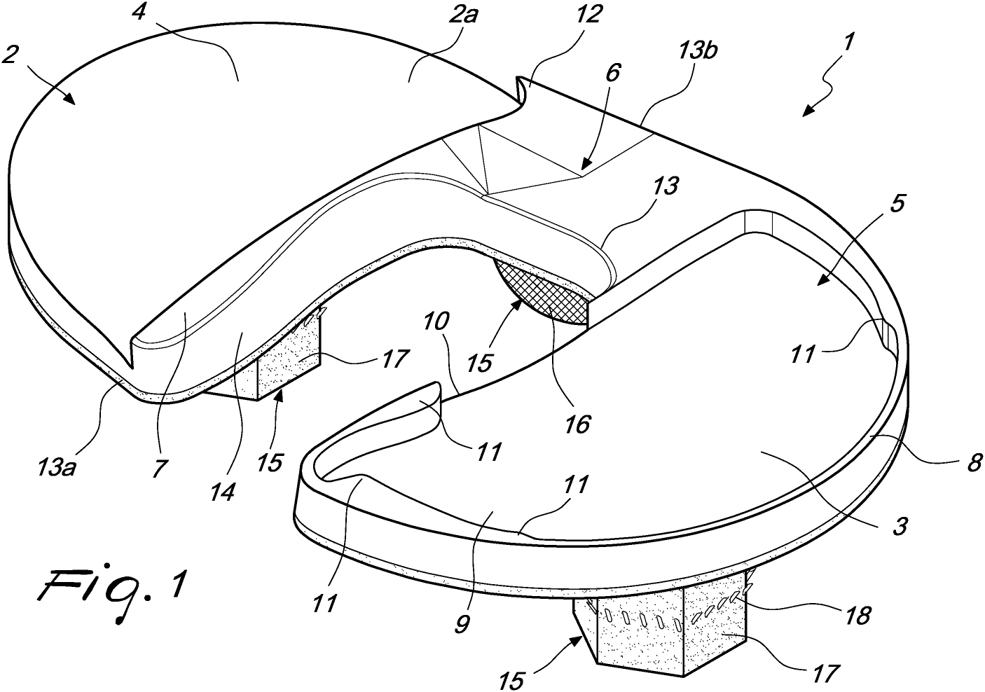

FIG. 1 is a perspective view from above of the tibial component of a total knee prosthesis, according to the disclosure;

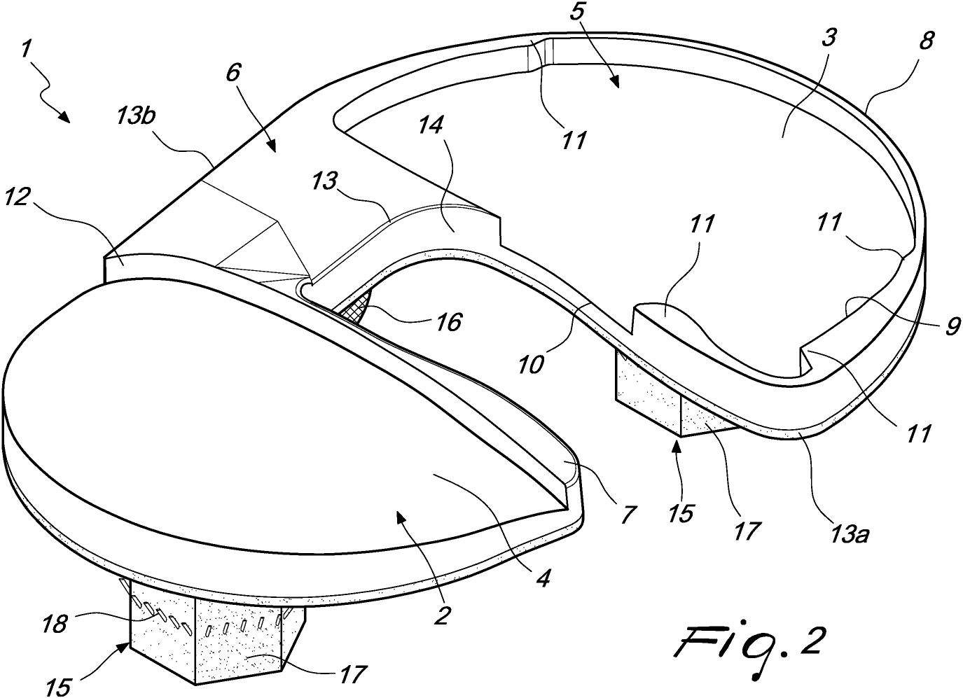

FIG. 2 is a different perspective view from above of the tibial component of a total knee prosthesis, according to the disclosure;

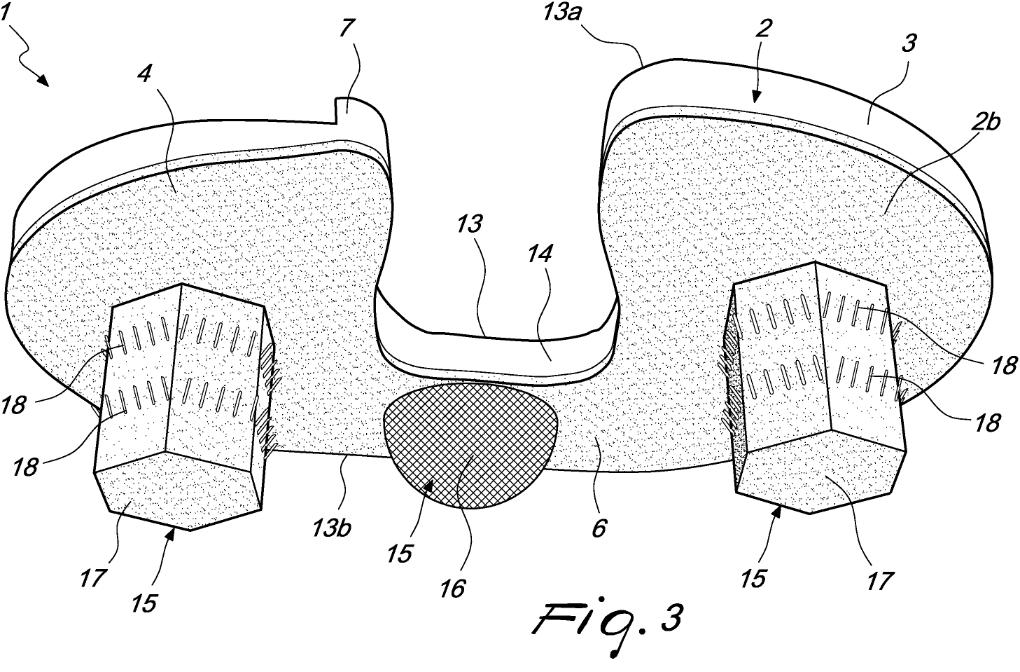

FIG. 3 is a perspective view from below of the tibial component of a total knee prosthesis, according to the disclosure;

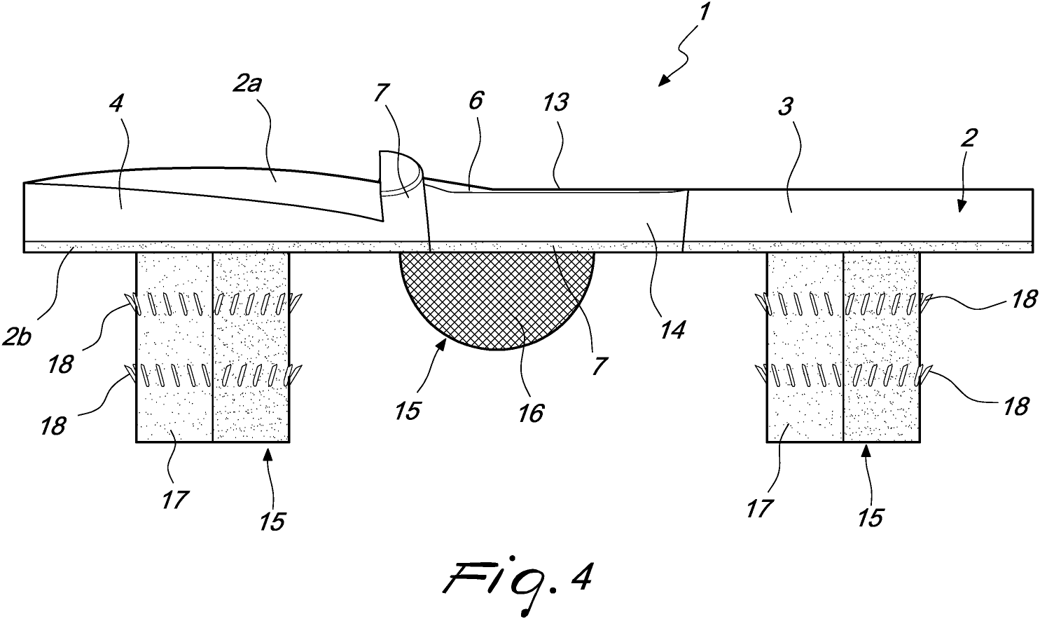

FIG. 4 is a front elevation view of the tibial component of a total knee prosthesis, according to the disclosure;

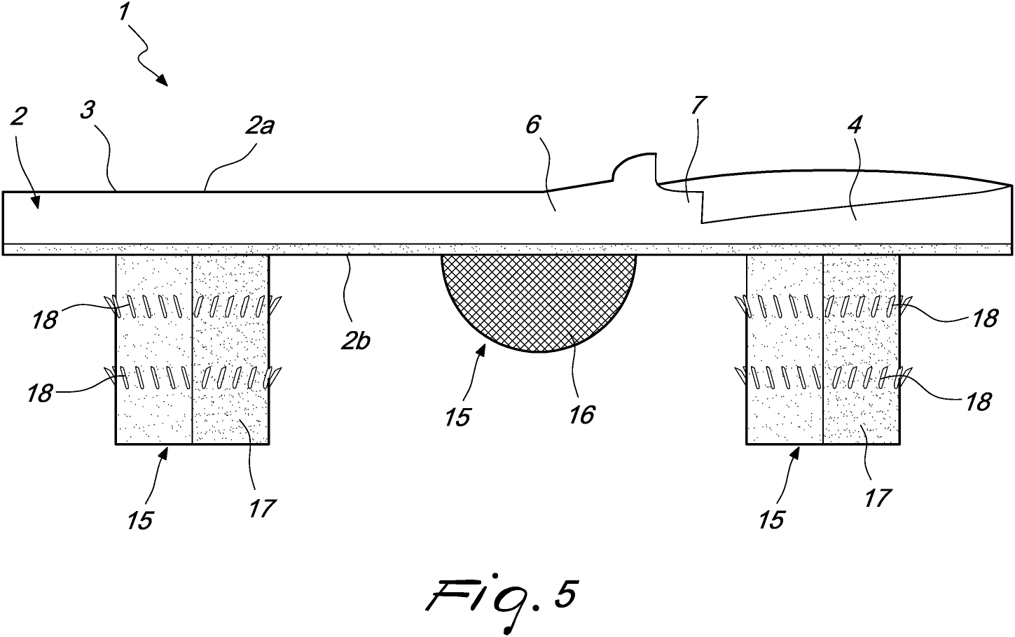

FIG. 5 is a rear elevation view of the tibial component of a total knee prosthesis, according to the disclosure;

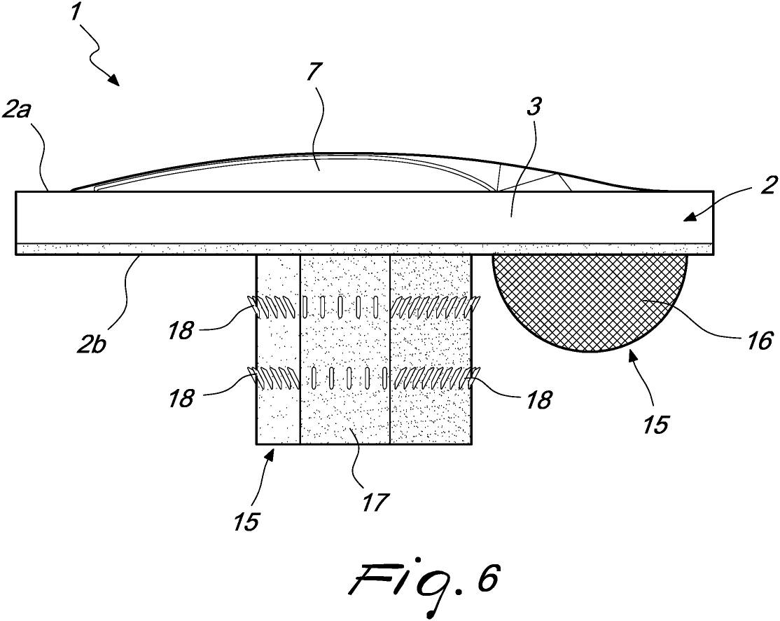

FIG. 6 is a side view from the right of the tibial component of a total knee prosthesis, according to the disclosure;

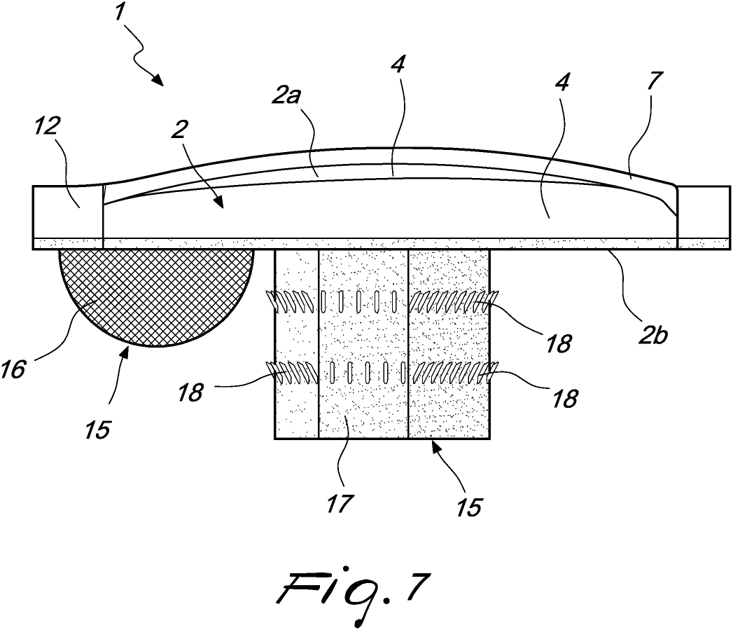

FIG. 7 is a side view from the left of the tibial component of a total knee prosthesis, according to the disclosure;

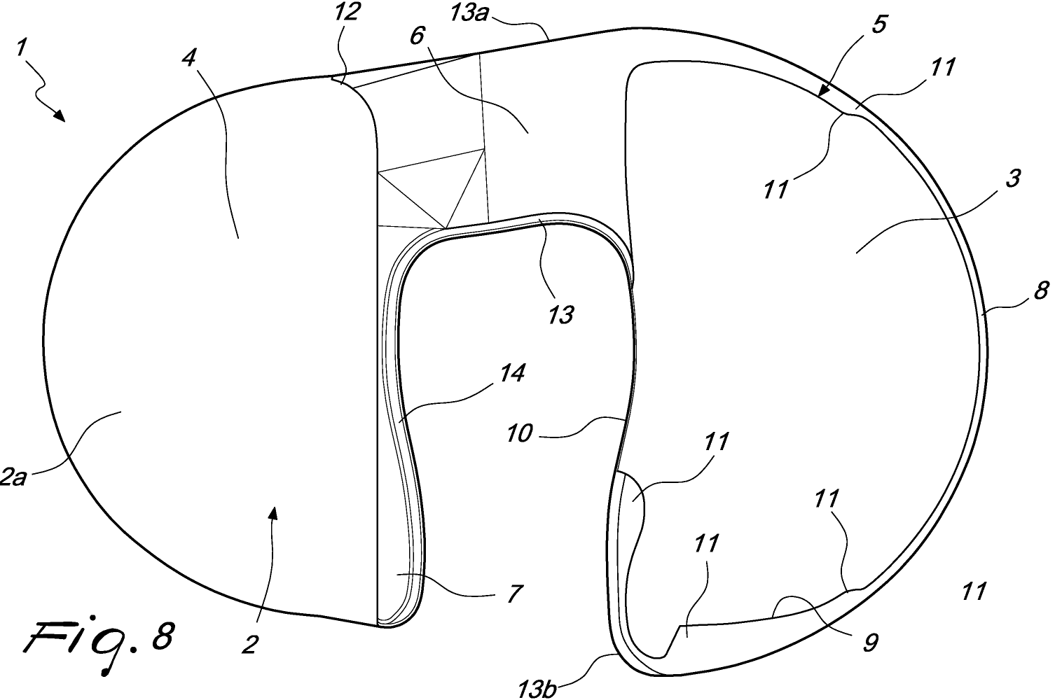

FIG. 8 is a plan view from above of the tibial component of a total knee prosthesis, according to the disclosure; and

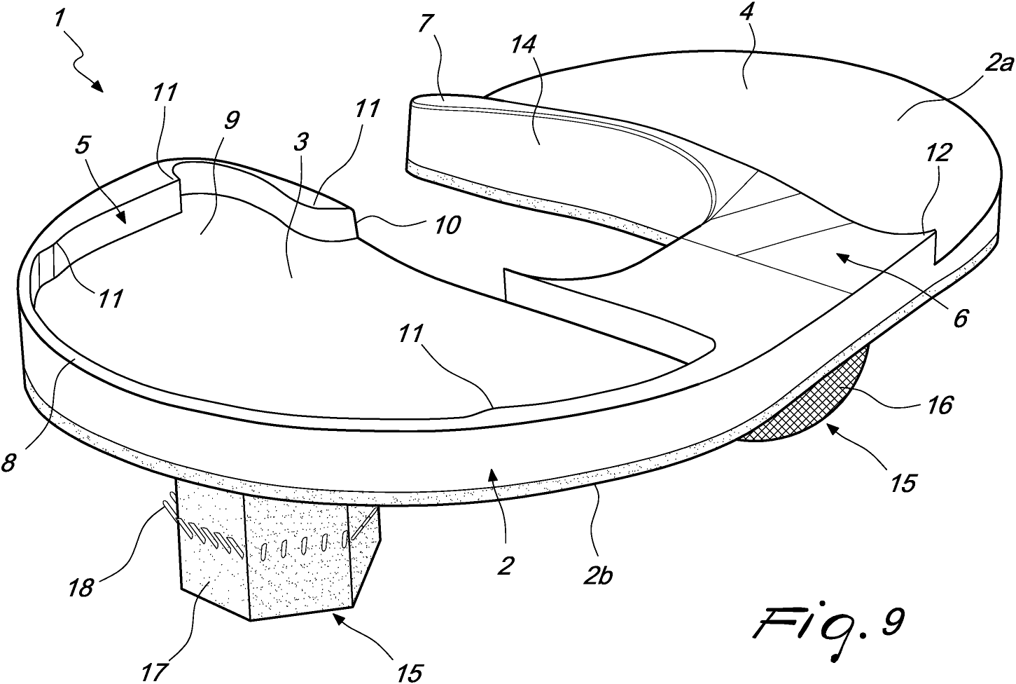

FIG. 9 is an additional perspective view from above of the tibial component of a total knee prosthesis, according to the disclosure.

### DETAILED DESCRIPTION OF THEDRAWINGS

A tibial component 1 of a total knee prosthesis according to the present disclosure comprises a base element 2 provided with a medial portion 3 and a lateral portion 4, which can be affixed on the proximal surface of the tibia.

It should be noted that in the medical jargon, such medial portion and such lateral portion are defined respectively as the medial compartment and the lateral compartment.

According to the disclosure, the base element 2 has a shape and dimensions that are complementary to those of at least one protective insert.

It should be noted from this point onward that the protective insert is designed to be interposed between the tibial component, according to the disclosure, and the femoral component of a total knee prosthesis, and to perform a function similar to that of a meniscus in a natural anatomical joint.

In particular, the medial portion 3 is provided with coupling means 5 by pressure to the insert (a configuration generally termed “fixed insert”).

By contrast, the at least one insert is resting on the lateral portion (a configuration generally termed “movable insert”); the lateral portion 4 therefore has no mechanism for fixing to the at least one insert.

In this manner, it is therefore possible to faithfully recreate the medial stability and the lateral mobility observed in the normal kinematic arrangement of healthy knee joints, thus restoring not only the anatomy but also the functionality of the compromised joint.

Therefore a total knee arthroplasty, according to the disclosure, is no longer seen as a solitary, single unit, but as the robust union of two separate unicondylar reconstructions; the tibial component 1, according to the disclosure, has in fact a specific shape and characteristics for each portion, medial 3 and lateral 4, as in a healthy knee.

This solution is therefore designed to obtain the same degree of satisfaction associated with a unicompartmental partial arthroplasty, markedly higher than a total arthroplasty.

Its shape in fact makes it possible to combine and enhance the advantages of both approaches (fixed insert and movable insert), while at the same time limiting the respective drawbacks.

It should furthermore be noted that the base element 2 can be coupled to a single insert; such insert can be coupled stably, with its own medial part, to the medial portion 3 and, with its own lateral part, be resting on the lateral portion 4.

Furthermore, the possibility is not ruled out that the base element 2 could be coupled to two respective inserts; a first insert can be coupled stably to the medial portion 3, and a second insert can be resting on the lateral portion 4.

According to an embodiment of particular utility and practicality, the lateral portion 4 can have, at the boundary region 6 with the medial portion 3, a guiding wall 7 in order to direct and drive the anteroposterior translational motion of the at least one insert.

It should furthermore be noted that the coupling means 5 can comprise a perimetric containment frame 8 which defines a contoured seat 9 for the stable accommodation of a respective complementarily-shaped protrusion of such at least one insert.

Such configuration ensures a solid and stable locking in place of the insert, at the medial portion 3, and at the same time allows an easy insertion by pressure of the insert by the surgeon.

In any case, the possibility is not ruled out that the perimetric frame 8 can have interruptions 10 and/or protrusions 11, in order to increase the contact surface between the insert and the base element 2, so minimizing any possible micro-movement between these elements.

According to a solution of particular effectiveness and efficiency, the upper face 2a of the lateral portion 4 can have a convex surface for accommodating the respective lateral part of the insert.

Such embodiment makes it possible to obtain a high level of physiological mobility, while at the same time recreating the natural convexity of the anatomical lateral compartment and ensuring however a safe and stable joint with minimum risk of dislocation of the insert.

As a consequence the guiding wall 7 can have the same convex profile/progression with the aim of giving the lateral part of the insert a continuous and constant interface, thus minimizing the risk of dislocation of said insert.

It should furthermore be noted that the guiding wall 7 can have a protrusion 12 at one of its ends in order to ensure an additional front retention against the movement of the insert.

According to a preferred embodiment, the tibial component 1 can comprise a recess 13 between the lateral portion 4 and the medial portion 3.

Such embodiment can be adopted for prostheses of the type called Bicruciate Retaining (BCR) total knee arthroplasty (TKA).

Such recess 13 can extend, in the anteroposterior direction, from the rear end 13a of such base element 2, up to a distance preferably comprised between 10 mm and 20 mm from the front end 13b of such base element 2.

Such recess 13 can have a progressively increasing width, and will make it possible to preserve both cruciate ligaments.

Such profile of the recess 13 will therefore make it possible to recreate the anatomical intercondyloid profile, which has a greater width at the front, at the anterior cruciate ligament, than at the rear, at the posterior cruciate ligament.

Such a profile of the recess 13, which therefore recreates the anatomical profile of a healthy knee, makes it possible to preserve the functionality of the cruciate ligaments.

The possibility is not ruled out furthermore that the recess 13 can have a greater depth at the medial portion 3 than at the lateral portion 4, in order to recreate even more faithfully the anatomical profile of a healthy knee.

It should furthermore be noted that the walls 14 of the recess 13 can be beveled/rounded and mutually inclined complementarily toward said recess 13 (at the minimum height such walls will be at the minimum distance from each other), in order to reduce the risk of prosthetic instability over time.

By contrast, the prostheses currently present on the market comprise tibial components with the recess having substantially vertical walls, which can therefore lead to an excessive buildup of stress and tension on said tibial component as well as, over time, possible micro/macro tearing of and damage to the anterior and posterior cruciate ligaments, and therefore as a consequence undermine the integrity and functionality, not only of the ligaments, but also of the prosthesis itself.

Indirectly, therefore, the tibial component 1, according to the disclosure, also reduces the risk of prosthetic instability over time.

The tibial component 1, according to the disclosure, can further comprise fixing elements 15 for fixing to a proximal surface of the tibia, which are defined at the lower face 2b of the base element 2.

Such elements 15 make it possible to minimize the instability of the tibial implant over time, which could otherwise determine the loss and therefore the failure of the prosthesis itself.

In particular, the fixing elements 15 can comprise a hemispherical protuberance 16 which is defined at the boundary region 6 between the lateral portion 4 and the medial portion 3 of the base element 2.

Such protuberance 16 will therefore make it possible to increase prosthetic stability and will prevent abnormal rotations of the tibial component 1 during normal movements performed by the joint.

The hemispherical shape of the protuberance 16 makes it possible to increase the bone contact surface and therefore the prosthetic stability and furthermore it simplifies curettage operations to provide a complementary furrow in the proximal part of the tibia by the surgeon during the operation.

Furthermore, the possibility is not ruled out that, on the surface of the protuberance 16, there can be furrows and/or grids, hollowed and/or in relief, which confer an effect of macro-roughness and porosity on the protuberance 16.

In a non-cemented fixing, such macro-roughness would be added to the micro-roughness of porous tibial materials such as for example Trabecular Metal (TM), thus increasing the phenomena of bone adhesion and prosthetic integration with adjacent tissues and, therefore, increasing the stability of the prosthesis.

Furthermore, if the tibial component 1 comprises the recess 13, the presence of the protuberance 16 will make it possible to thicken, stiffen and therefore strengthen the boundary region 6 of the base element 2.

It is known in fact that the most fragile region of a tibial prostheses of the type called Bicruciate Retaining (BCR) total knee arthroplasty (TKA), is the boundary region 6, constituted by a narrow bridge that connects the two portions, lateral 4 and medial 3, and which can be subject to fracturing over time.

The possibility is furthermore not ruled out that the fixing elements 15 can comprise, in addition to or in substitution of the hemispherical protuberance 16, two pins 17 which are respectively defined at the lateral portion 4 and at the medial portion 3.

It should be noted that such pins 17 could have a hexagonal shape.

The hexagonal shape in fact makes it possible to obtain a stable coupling with the circular bone cavities of the tibia that are intended to receive them, precisely by virtue of the discrepancy between the two shapes.

It should furthermore be noted that the pins 17 can have a length comprised between 10 and 20 mm and the pin of the medial portion 3 can be in a position further to the rear than the pin of the lateral portion 4, thus ensuring a correct placement inside the tibial epiphysis with the pins 17 interfacing with the area of greatest bone density, aligned with the condylar load.

In this manner it is possible to safeguard against the risk of protrusion and release of the pins 17 beyond the cortical bone surface layer.

Such pins 17 can have on their lateral surface 14 at least one row of spikes 18, which are directed toward the lower face 2b of the base element 2, in order to ensure optimum stability of the tibial component 1.

The upward orientation of the tips 18 will ensure that the tips flex, during the operations to insert the pins 17 into the bone cavity of the proximal part of the tibia, and on the contrary impede the removal of the implant or an unintentional detachment of the implant.

In fact, once inserted, the tips 18, by anchoring to the trabecular structures of the adjacent bone tissue, ensure an exceptional immediate stability of the tibial implant, which represents the principal critical point of operations of total arthroplasty of knees in which cement is not used to fix the prosthesis to the bone tissue of the patient.

In a non-cemented fixing, during an operation to revision the prosthesis, it would therefore be possible to leave the fixing elements 15 (the protuberance 16 and the pins 17) inside the proximal part of the tibia, removing only the base element 2, through an osteotomy, in that such fixing elements, in a non-cemented fixing, would be completely physically and biologically embedded in the bone reticulum and would not represent a problem for the organism.

The tibial component of the new, revisioned prostheses could then be simply positioned above such elements 15 (with the stem of the new, revisioned prostheses interposed between said fixing elements 15).

In an embodiment of significant practical interest, the medial portion 3 can have a greater width than the lateral portion 4, in order to maximize bone coverage and minimize the risk of tibial rotational misalignment.

It is therefore evident that the medial portion 3 can be superimposed on bone surfaces of greater breadth in an anteroposterior direction (in medical jargon, that means it can cover positions further to the front and to the rear) with respect to the lateral portion 4.

It should furthermore be noted that the difference between the width (anteroposterior dimension) of the medial portion 3 and of the lateral portion 4 may become more marked as one moves toward the center of the joint, i.e. toward the boundary region 6.

It should be noted that the radius (of curvature) of the medial portion 3 is substantially symmetrical, with respect to the radius of the lateral portion 4 which, by contrast, increases towards the rear.

It should furthermore be noted that the base element 2 can have shape and dimensions that are defined on the basis of proximal tibial morphological analyses attributable in the literature to Dr. Geoffrey et al.

The adoption therefore of a profile that is anatomical/asymmetrical (between the medial portion 3 and the lateral portion 4), which follows more faithfully the proximal tibial bone profile of healthy knee joints, makes it possible to maximize bone coverage and cortical support while at the same time minimizing the risk of the absence of coverage where necessary and of the presence of coverage where, by contrast, it is not necessary.

Furthermore, the base element 2 can have a thickness preferably comprised between 2.3 mm and 2.8 mm in order to minimize the instability of the prosthesis itself, thus making it possible also to lessen bone resorption around the prostheses and therefore minimize the unwanted phenomenon known in the medical jargon as stress-shielding.

The prosthesis is therefore more firmly embedded in the bone tissue of the patient, consequently limiting episodes of instability and loss of the prosthesis.

Finally, by reducing the thickness of the base element 2, it is possible to increase the minimum thickness of the polyethylene inserts, with a consequent reduction in the wear of the polyethylene.

It should be noted that the adoption of the tibial component 1, according to the disclosure, is independent of the type of total knee prosthesis used.

It can therefore positively be used for any type of Total Knee Arthroplasty (TKA).

The adoption therefore of a hybrid tibial component 1, according to the disclosure, with a medial, fixed insert and a lateral, movable insert, applied to the total knee arthroplasty (TKA), makes it possible to definitively recreate the anatomy and functionality of the knee joint, without requiring any kind of compromise, between mobility and stability, functionality and anatomy, congruence and permittivity.

It should be emphasized that the advantage of the innovation lies, in addition to in the hybrid tibial component 1, also in the shape of the recess 13 (with progressively increasing width and with walls 14 that are beveled/rounded and mutually inclined complementarily toward said recess 13) and in the adoption of the fixing elements 15 (the presence of the hemispherical protuberance 16 and of the tips 16 defined on the pins 17).

Advantageously, the tibial component 1 of a total knee prosthesis makes it possible to recreate the physiological medial stability and the physiological lateral mobility observed in the normal kinematic arrangement of healthy knee joints.

Effectively, the tibial component 1 of a total knee prosthesis makes it possible to reduce to the minimum the risk of dislocation of the at least one insert.

Profitably, the tibial component 1 according to the disclosure makes it possible to reduce to the minimum the risk of prosthetic instability over time.

Conveniently, the tibial component 1 of a total knee prosthesis makes it possible to reduce to the minimum phenomena of wear of the total knee prosthesis.

Conveniently, the tibial component 1 of a total knee prosthesis is of low cost, easily and practically implemented and safe in use.

The disclosure, thus conceived, is susceptible of numerous modifications and variations, all of which are within the scope of the appended claims. Moreover, all the details may be substituted by other, technically equivalent elements.

In the embodiments illustrated, individual characteristics shown in relation to specific examples may in reality be interchanged with other, different characteristics, existing in other embodiments.

In practice, the materials employed, as well as the dimensions, may be any according to requirements and to the state of the art.

What is claimed is:

1. A tibial component of a total knee prosthesis, comprising a base element provided with a medial portion and a lateral portion, configured to be fixed on the proximal surface of the tibia, wherein said base element has a shape and dimensions that are complementary to those of said at least one protective insert, said medial portion being provided with coupling means configured for coupling by pressure to said at least one insert, the at least one insert resting on said lateral portion.

2. The tibial component according to claim 1, wherein said lateral portion has, at a boundary region with said medial portion, a guiding wall configured to direct and drive the anteroposterior translational motion of the at least one insert.

3. The tibial component according to claim 1, wherein said coupling means comprise a perimeter containment frame which defines a contoured seat for the stable accommodation of a respective complementarily-shaped protrusion of the at least one insert.

4. The tibial component according to claim 1, wherein an upper face of said lateral portion has a convex surface for accommodating at least one insert; configured to minimize the risk of dislocation of said insert.

5. The tibial component according to claim 1, further comprising a recess between the lateral portion and the medial portion which extends, in an anteroposterior direction, from a rear end of said base element toward the front end of said base element, said recess having a progressively increasing width.

6. The tibial component according to claim 5, wherein walls of said recess are beveled/rounded and mutually inclined complementarily toward said recess; configured to reduce a risk of prosthetic instability over time.

7. The tibial component according to claim 1, further comprising fixing elements for fixing to the proximal surface of the tibia, which are defined at a lower face of said base element.

8. The tibial component according to claim 7, wherein said fixing elements comprise a hemispherical protuberance which is defined at a boundary portion between said lateral portion and said medial portion of said base element configured to increase prosthetic stability and prevent abnormal rotations of the tibial component during normal movements performed by a joint.

9. The tibial component according to claim 7, wherein said fixing elements comprise two pins which are respectively defined at said lateral portion and said medial portion, said pins having, on a lateral surface, at least one row of spikes which are directed toward said lower face of said base element; configured to ensure optimum stability of said tibial component.

10. The tibial component according to claim 1, wherein said base element has an asymmetrical anatomical profile, said medial portion having a greater width than said lateral portion configured to maximize bone coverage and minimize a risk of tibial rotational misalignment.

11. The tibial component according to claim 1, wherein said base element has a thickness comprised between 2.3 mm and 2.8 mm configured to minimize instability of the total knee prosthesis.
